(19) United States
(12) Patent Application Publication
(10) Pub. No.: US 2025/0122286 A1
(43) Pub. Date: Apr. 17, 2025

(54) ANTIBODY OPTIMIZATION

(71) Applicant: Eli Lilly and Company, Indianapolis, IN (US)

(72) Inventors: Brian David BOWES, Indianapolis, IN (US); David John STOKELL, Indianapolis, IN (US)

(21) Appl. No.: 18/293,836

(22) PCT Filed: Aug. 5, 2022
(86) PCT No.: PCT/US2022/074581

§ 371 Date: Jan. 31, 2024

Related U.S. Application Data
(60) Provisional application No. 63/276,145, filed on Nov. 5, 2021.
(60) Provisional application No. 63/229,772, filed on Aug. 5, 2021.

(51) Int. Cl. C07K 16/28

(52) U.S. Cl. CPC C07K 16/283; C07K 2317/522; C07K 2317/524; C07K 2317/526; C07K 2317/565; C07K 2317/90; C07K 2317/92

(57) ABSTRACT

The present disclosure relates to methods of developing, designing, and producing therapeutic antibodies to improve developability of an antibody or antibody drug conjugate.

The present invention is in the field of medicine. More particularly, the present invention provides methods of developing, designing, and producing therapeutic antibodies and conjugates thereof. In particular embodiments, methods disclosed herein provide an improvement in developability of an antibody or antibody drug conjugate, for example a reduction in viscosity and/or aggregation.

Therapeutic antibodies have been developed to treat numerous diseases. Common challenges faced in development of therapeutic antibodies include for example, high viscosity, aggregation, low solubility, half-life extension, and immunogenicity. Some of these challenges can impact stability of the therapeutic antibody which can be further exacerbated when formulating for delivery. While therapeutic antibodies are commonly administered via intravenous (IV) infusion, subcutaneous administration provides several advantages over IV, such as more effective pharmacokinetic profiles, option for self-administration which is important for chronic diseases requiring frequent long-term dosing. Furthermore, subcutaneous administration enables ready-to-use pre-filled delivery devices providing, greater patient comfort, reduced treatment time, potentially improving compliance, treatment outcome, and reduced costs.

However, the development of therapeutic antibody formulations suitable for subcutaneous administration poses multiple challenges, particularly, the limited volume available for subcutaneous administration necessitates for highly concentrated antibody solutions. Highly concentrated antibody solutions can lead to increased viscosity, and/or aggregation. High viscosity can increase injection time and pain at the injection site, impacting patient compliance, and can also destabilize the drug substance during bioprocessing, impacting pharmacokinetic profiles, biological activity, increased manufacturing costs, and potentially preventing a drug from moving forward in development. Similarly, aggregation poses a major issue because the trend toward high-concentration solutions increases the likelihood of protein-protein interactions, which in turn favors aggregation.

Approaches to reducing viscosity of antibody therapeutics have been studied. Such methods include, modifying formulation conditions, such as, adjusting pH, buffer conditions, ionic strength, or adding other excipients. However, these methods have proven to be limited in addressing the viscosity issues for a broader range of antibody therapeutics. For example, in some instances, modifying formulation conditions may require extra process steps in manufacturing, impact stability, aggregation, immunogenicity, and/or pharmacokinetic profiles, all of which can be costly and compromise the development of the antibody therapeutic. Additionally, attempts to optimize amino acid sequences in the variable region of an IgG1 antibody to reduce viscosity was reported (Tomar at al., Mabs 2016, 8 (2): 216-228). However, such methods are limited to the particular antibody variable region being optimized. As such, there remains a need for additional methods of reducing viscosity of therapeutic antibodies at high concentrations, where the methods are applicable across a broad spectrum of antibody therapeutics, such as IgG1, IgG2, IgG3, or IgG4 antibodies, and are not limited to the particular therapeutic of interest, and where such methods do not negatively impact affinity, stability, aggregation, immunogenicity, or biological functions, and do not require costly process or formulation changes.

Approaches to improving half-life for peptides and small proteins such as fusing a peptide to an Fc portion of an antibody for time extension have been studied. In some instances, such methods involve expression of smaller proteins or peptides on the N or C terminus of an antibody Fc comprised of the hinge, CH2 and CH3 domains. Fusions of this type can improve half-life by, for example, decreasing renal clearance. However, such fusions can result in increase in viscosity or decreases in pH stability of the fusion protein, which can require significant formulation optimization or further engineering of the peptide or protein sequence.

Accordingly, the present disclosure addresses one or more of the above needs by providing alternative compositions and methods for improving developability of antibody therapeutics, such as by reducing viscosity and/or improving half-life of an antibody or antibody drug conjugate at high concentrations. Specifically, the present disclosure provides compositions and methods comprising modifying the constant region of an antibody, wherein the methods do not negatively impact antibody affinity, aggregation, and/or stability, or biological functions such as effector function, or do not require costly changes in formulation and downstream manufacturing processes. More particularly, the present disclosure provides compositions and methods comprising amino acid substitutions in the IgG heavy chain constant domains of an antibody that may be applicable across a wide platform of antibody therapeutics such as IgG2, IgG3, or IgG4 antibody therapeutics.

Accordingly, in particular embodiments, the present disclosure provides an antibody having a modified human IgG heavy chain (HC) constant region comprising one or more of the following amino acid substitutions compared to the wild-type human IgG heavy chain constant region: E137G, D203N, Q274K, Q355R, E419Q, (all positions numbered according to EU numbering). Accordingly, in particular embodiments, the present disclosure provides an antibody comprising a human IgG HC constant region comprising a constant heavy chain 1 (CH1) domain, a constant heavy chain 2 (CH2) domain, and a constant heavy chain 3 (CH3) domain, and a human IgG light chain (LC) constant region, wherein the human IgG HC constant region comprises: a lysine at amino acid residue 274 (EU numbering) of the CH2 domain; an arginine at amino acid residue 355 (EU numbering) of the CH3 domain; a glutamine at amino acid residue 419 (EU numbering) of the CH3 domain; a glycine at amino acid residue 137 (EU numbering) of the CH1 domain; an asparagine at amino acid residue 203 (EU numbering) of the CH1 domain; a lysine at amino acid residue 274 (EU numbering) of the CH2 domain and an arginine at amino acid residue 355 (EU numbering) of the CH3 domain; a lysine at amino acid residue 274 (EU numbering) of the CH2 domain and a glutamine at amino acid residue 419 (EU numbering) of the CH3 domain; an arginine at amino acid residue 355 (EU numbering) of the CH3 domain and a glutamine at amino acid residue 419 (EU numbering) of the CH3 domain; a lysine at amino acid residue 274 (EU numbering) of the CH2 domain and a glycine at amino acid residue 137 (EU numbering) of the CH1 domain; a lysine at amino acid residue 274 (EU numbering) of the CH2 domain and an asparagine at amino acid residue 203 (EU numbering) of the CH1 domain; an arginine at amino acid residue 355 (EU numbering) of the CH3 domain and a glycine at amino acid residue 137 (EU numbering) of the CH1 domain; an arginine at amino acid residue 355 (EU numbering) of the CH3 domain and an asparagine at amino acid residue 203 (EU numbering) of the CH1 domain; a glutamine at amino acid residue 419 (EU numbering) of the CH3 domain and a glycine at amino acid residue 137 (EU numbering) of the CH1 domain; a glutamine at amino acid residue 419 (EU numbering) of the CH3 domain and an asparagine at amino acid residue 203 (EU numbering) of the CH1 domain; a glycine at amino acid residue 137 (EU numbering) of the CH1 domain and an asparagine at amino acid residue 203 (EU numbering) of the CH1 domain; a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain; a lysine at amino acid residue 274 (EU numbering) of the CH2 domain, an arginine at amino acid residue 355 (EU numbering) of the CH3 domain, and a glycine at amino acid residue 137 (EU numbering) of the CH1 domain; a lysine at amino acid residue 274 (EU numbering) of the CH2 domain, an arginine at amino acid residue 355 (EU numbering) of the CH3 domain, and an asparagine at amino acid residue 203 (EU numbering) of the CH1 domain; a lysine at amino acid residue 274 (EU numbering) of the CH2 domain, a glycine at amino acid residue 137 (EU numbering) of the CH1 domain, and an asparagine at amino acid residue 203 (EU numbering) of the CH1 domain; a lysine at amino acid residue 274 (EU numbering) of the CH2 domain, a glutamine at amino acid residue 419 (EU numbering) of the CH3 domain, and a glycine at amino acid residue 137 (EU numbering) of the CH1 domain; a lysine at amino acid residue 274 (EU numbering) of the CH2 domain, a glutamine at amino acid residue 419 (EU numbering) of the CH3 domain, and an asparagine at amino acid residue 203 (EU numbering) of the CH1 domain; an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain; an arginine at amino acid residue 355 (EU numbering) of the CH3 domain, a glutamine at amino acid residue 419 (EU numbering) of the CH3 domain, an asparagine at amino acid residue 203 (EU numbering) of the CH1 domain; an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain; a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain; a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and a glycine at amino acid residue 137 (EU numbering) of the CH1 domain; a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and an asparagine at amino acid residue 203 (EU numbering) of the CH1 domain; a lysine at amino acid residue 274 (EU numbering) of the CH2 domain, an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain; a lysine at amino acid residue 274 (EU numbering) of the CH2 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain; an arginine at amino acid residue 355 (EU numbering) of the CH3 domain, a glutamine at amino acid residue 419 (EU numbering) of the CH3 domain; a glycine at amino acid residue 137 (EU numbering) of the CH1 domain, and an asparagine at amino acid residue 203 (EU numbering) of the CH1 domain; or a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain.

In particular embodiments, the present disclosure provides an antibody having a modified human IgG heavy chain (HC) constant region comprising two or more of the following amino acid substitutions compared to the wild-type human IgG heavy chain constant region: E137G, D203N, Q274K, Q355R, E419Q, R409K (all positions numbered according to EU numbering). Accordingly, in particular embodiments, the present disclosure provides an antibody having a modified human IgG heavy chain (HC) constant region comprising three or more of the following amino acid substitutions compared to the wild-type human IgG heavy chain constant region: E137G, D203N, Q274K, Q355R, E419Q, R409K (all positions numbered according to EU numbering).

According to some embodiments, the present disclosure provides an antibody comprising a human IgG HC constant region comprising a CH1, CH2, and a CH3 domain, and a human IgG light chain constant region, wherein the human IgG HC constant region comprises: a lysine at amino acid residue 274 (EU numbering) of the CH2 domain; an arginine at amino acid residue 355 (EU numbering) of the CH3 domain; a glutamine at amino acid residue 419 (EU numbering) of the CH3 domain; a lysine at amino acid residue 274 (EU numbering) of the CH2 domain and an arginine at amino acid residue 355 (EU numbering) of the CH3 domain; a lysine at amino acid residue 274 (EU numbering) of the CH2 domain and a glutamine at amino acid residue 419 (EU numbering) of the CH3 domain; an arginine at amino acid residue 355 (EU numbering) of the CH3 domain and a glutamine at amino acid residue 419 (EU numbering) of the CH3 domain; or a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

According to further embodiments, the present disclosure provides an antibody comprising a human IgG HC constant region comprising a CH1, CH2, and a CH3 domain, and a human IgG light chain constant region, wherein the human IgG heavy chain constant region comprises, a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

In further embodiments, the antibodies of the present disclosure comprise a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3.

In some embodiments the antibody of the present disclosure, has a human IgG2, human IgG3, or a human IgG4 isotype. In some embodiments, the antibody of the present disclosure has a human IgG4 isotype. In some embodiments, the antibodies as described herein comprise a modified human IgG4 hinge region comprising a S228P mutation (EU numbering), which reduces the IgG4 Fab-arm exchange in vivo (see Labrijn, et al., Nat. Biotechnol. 2009, 27 (8): 767). In some embodiments, the antibodies described herein have a modified IgG4 Fc region having reduced or eliminated Fc effector functions (i.e., IgG4 Fc-effector null). Such antibodies, comprise for example, amino acid residues modifications F234A and/or L235A in the IgG4 Fc region to reduce binding to the FcγR (all residues numbered according to EU numbering). In some embodiments, the antibodies described herein comprise an IgG4 hinge region comprising a proline at residue 228, and an IgG4 Fc region comprising an alanine at residues 234 and 235 (all residues numbered according to EU numbering).

In some embodiment, the antibodies of the present disclosure comprise a human IgG heavy chain constant region comprising any one of SEQ ID NOs: 8, 9, 10, 12, 13, or 16.

In some embodiments, the present disclosure also provides antibody drug conjugates comprising an antibody disclosed herein.

In some embodiments, the antibody of the present disclosure has a modified human IgG HC constant region which reduces viscosity and/or improves stability of the antibody. In some embodiments, the antibody or antibody drug conjugate of the present disclosure has reduced viscosity when compared to a wild-type antibody comprising a wild-type human IgG heavy chain constant region. In further embodiments of the present disclosure, the viscosity of the antibody or antibody drug conjugate is reduced by about 25 percent to about 80 percent when compared to the wild-type antibody. In some embodiments, the antibody of the present disclosure has a modified human IgG4 HC constant region comprising one or more of the following amino acid substitutions compared to the wild-type human IgG4 HC constant region: E137G, D203N, Q274K, Q355R, E419Q, (all positions numbered according to EU numbering). In some embodiments the antibody of the present disclosure, has a modified human IgG4 HC constant region comprising one or more of the following amino acid substitutions compared to the wild-type human IgG4 HC constant region: Q274K, Q355R, E419Q. In some embodiments, the antibody or antibody drug conjugate of the present disclosure has a modified human IgG4 HC constant region which reduces viscosity of the antibody by about 5%, about 10%, about 15%, about 20%, about 25%, about 30%, about 35%, about 40%, about 45%, about 50%, about 55%, about 60%, about 65%, about 70%, about 80%, about 85%, about 90%, or about 100%. In some embodiments the antibody of the present disclosure has a therapeutically acceptable viscosity for delivery to a patient. In such embodiments the viscosity is below 14 cP, below 12 cP, or below 10 cP. In some embodiments the antibody is administered by subcutaneous administration or by intravenous administration.

In some embodiments the antibody of the present disclosure, has a modified human IgG2 HC constant region comprising one or more of the following amino acids residues: E137G, D203N, Q274K (all positions numbered according to EU Numbering). In some embodiments the antibody of the present disclosure, has a modified human IgG3 HC constant region comprising amino acid residue Q274K (numbered according to EU Numbering). In such embodiments, the human IgG2 and or human IgG3 antibody has reduced viscosity when compared to the respective wild-type antibody comprising a wild-type human IgG2 or human IgG3 heavy chain constant region.

In some embodiments, the present disclosure provides a method of reducing viscosity of a human IgG4 antibody comprising, generating a variant of the human IgG4 antibody comprising a modified human IgG4 heavy chain constant region comprising a CH1, CH2, and a CH3 domain, and a human IgG light chain constant region, wherein the modified human IgG4 heavy chain constant region comprises the following amino acid residues, a lysine at amino acid residue 274 (EU numbering) of the CH2 domain; an arginine at amino acid residue 355 (EU numbering) of the CH3 domain; a glutamine at amino acid residue 419 (EU numbering) of the CH3 domain; a glycine at amino acid residue 137 (EU numbering) of the CH1 domain; an asparagine at amino acid residue 203 (EU numbering) of the CH1 domain; a lysine at amino acid residue 274 (EU numbering) of the CH2 domain and an arginine at amino acid residue 355 (EU numbering) of the CH3 domain; a lysine at amino acid residue 274 (EU numbering) of the CH2 domain and a glutamine at amino acid residue 419 (EU numbering) of the CH3 domain; an arginine at amino acid residue 355 (EU numbering) of the CH3 domain and a glutamine at amino acid residue 419 (EU numbering) of the CH3 domain; a lysine at amino acid residue 274 (EU numbering) of the CH2 domain and a glycine at amino acid residue 137 (EU numbering) of the CH1 domain; a lysine at amino acid residue 274 (EU numbering) of the CH2 domain and an asparagine at amino acid residue 203 (EU numbering) of the CH1 domain; an arginine at amino acid residue 355 (EU numbering) of the CH3 domain and a glycine at amino acid residue 137 (EU numbering) of the CH1 domain; an arginine at amino acid residue 355 (EU numbering) of the CH3 domain and an asparagine at amino acid residue 203 (EU numbering) of the CH1 domain; a glutamine at amino acid residue 419 (EU numbering) of the CH3 domain and a glycine at amino acid residue 137 (EU numbering) of the CH1 domain; a glutamine at amino acid residue 419 (EU numbering) of the CH3 domain and an asparagine at amino acid residue 203 (EU numbering) of the CH1 domain; a glycine at amino acid residue 137 (EU numbering) of the CH1 domain and an asparagine at amino acid residue 203 (EU numbering) of the CH1 domain; a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain; a lysine at amino acid residue 274 (EU numbering) of the CH2 domain, an arginine at amino acid residue 355 (EU numbering) of the CH3 domain, and a glycine at amino acid residue 137 (EU numbering) of the CH1 domain; a lysine at amino acid residue 274 (EU numbering) of the CH2 domain, an arginine at amino acid residue 355 (EU numbering) of the CH3 domain, and an asparagine at amino acid residue 203 (EU numbering) of the CH1 domain; a lysine at amino acid residue 274 (EU numbering) of the CH2 domain, a glycine at amino acid residue 137 (EU numbering) of the CH1 domain, and an asparagine at amino acid residue 203 (EU numbering) of the CH1 domain; a lysine at amino acid residue 274 (EU numbering) of the CH2 domain, a glutamine at amino acid residue 419 (EU numbering) of the CH3 domain, and a glycine at amino acid residue 137 (EU numbering) of the CH1 domain; a lysine at amino acid residue 274 (EU numbering) of the CH2 domain, a glutamine at amino acid residue 419 (EU numbering) of the CH3 domain, and an asparagine at amino acid residue 203 (EU numbering) of the CH1 domain; an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain; an arginine at amino acid residue 355 (EU numbering) of the CH3 domain, a glutamine at amino acid residue 419 (EU numbering) of the CH3 domain, an asparagine at amino acid residue 203 (EU numbering) of the CH1 domain; an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain; a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain; a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and a glycine at amino acid residue 137 (EU numbering) of the CH1 domain; a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and an asparagine at amino acid residue 203 (EU numbering) of the CH1 domain; a lysine at amino acid residue 274 (EU numbering) of the CH2 domain, an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain; a lysine at amino acid residue 274 (EU numbering) of the CH2 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain; an arginine at amino acid residue 355 (EU numbering) of the CH3 domain, a glutamine at amino acid residue 419 (EU numbering) of the CH3 domain; a glycine at amino acid residue 137 (EU numbering) of the CH1 domain, and an asparagine at amino acid residue 203 (EU numbering) of the CH1 domain; or a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain.

In some embodiments, the present disclosure provides a method of reducing viscosity of a human IgG4 antibody comprising, generating a variant of the human IgG4 antibody comprising a modified human IgG4 heavy chain constant region comprising a CH1, CH2, and a CH3 domain, and a human IgG light chain constant region, wherein the modified human IgG4 heavy chain constant region comprises the following amino acid residues, a lysine at amino acid residue 274 (EU numbering) of the CH2 domain; an arginine at amino acid residue 355 (EU numbering) of the CH3 domain; a glutamine at amino acid residue 419 (EU numbering) of the CH3 domain; a lysine at amino acid residue 274 (EU numbering) of the CH2 domain and an arginine at amino acid residue 355 (EU numbering) of the CH3 domain; a lysine at amino acid residue 274 (EU numbering) of the CH2 domain and a glutamine at amino acid residue 419 (EU numbering) of the CH3 domain; an arginine at amino acid residue 355 (EU numbering) of the CH3 domain and a glutamine at amino acid residue 419 (EU numbering) of the CH3 domain; or a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

In some embodiments, the present disclosure provides a method of reducing viscosity of a human IgG4 antibody comprising, generating a variant of the human IgG4 antibody comprising a modified human IgG4 heavy chain constant region comprising a CH1, CH2, and a CH3 domain, and a human IgG light chain constant region, wherein the modified human IgG4 heavy chain constant region comprises the following amino acid residues: a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

In some embodiments, nucleic acid sequences encoding the antibodies of the present disclosure are provided. In some embodiments of the present disclosure, nucleic acids encoding a HC or LC of the antibodies are provided. In some embodiments of the present disclosure, nucleic acids encoding a VH or VL of the antibodies are provided. Some embodiments of the present disclosure, provide vectors comprising a nucleic acid sequence encoding an antibody HC or LC. Some embodiments of the present disclosure, provide vectors comprising a nucleic acid sequence encoding an antibody VH or VL.

Nucleic acids of the present disclosure may be expressed in a host cell, for example, after the nucleic acids have been operably linked to an expression control sequence. Expression control sequences capable of expression of nucleic acids to which they are operably linked are well known in the art. An expression vector may include a sequence that encodes one or more signal peptides that facilitate secretion of the polypeptide(s) from a host cell. Expression vectors containing a nucleic acid of interest (e.g., a nucleic acid encoding a heavy chain or light chain of an antibody) may be transferred into a host cell by well-known methods, e.g., stable or transient transfection, transformation, transduction or infection. Additionally, expression vectors may contain one or more selection markers, e.g., tetracycline, neomycin, and dihydrofolate reductase, to aide in detection of host cells transformed with the desired nucleic acid sequences.

In another aspect, provided herein are cells, e.g., host cells, comprising the nucleic acids, vectors, or nucleic acid compositions described herein. A host cell may be a cell stably or transiently transfected, transformed, transduced or infected with one or more expression vectors expressing all or a portion of an antibody described herein. In some embodiments, a host cell may be stably or transiently transfected, transformed, transduced or infected with an expression vector expressing HC and LC polypeptides of an antibody of the present disclosure. In some embodiments, a host cell may be stably or transiently transfected, transformed, transduced, or infected with a first vector expressing HC polypeptides and a second vector expressing LC polypeptides of an antibody described herein. Such host cells, e.g., mammalian host cells, can express the antibodies that specifically bind human IL-4Rα as described herein. Mammalian host cells known to be capable of expressing antibodies include CHO cells, HEK293 cells, COS cells, and NS0 cells.

In some embodiments, the cell, e.g., host cell, comprises a first vector and a second vector comprising the nucleic acid sequences encoding an antibody as provided herein. In further embodiments, the host cell is a mammalian cell.

The present disclosure further provides a process for producing an antibody or antibody binding fragments thereof as described herein, by culturing the host cell described above, e.g., a mammalian host cell, under conditions such that the antibody is expressed and recovering the expressed antibody from the culture medium. The culture medium, into which an antibody has been secreted, may be purified by conventional techniques. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

The present disclosure further provides antibodies or antibody binding fragments thereof produced by any of the processes described herein.

In another aspect, provided herein are pharmaceutical compositions comprising an antibody, nucleic acid, or vector described herein. Such pharmaceutical compositions can also comprise one or more pharmaceutically acceptable excipient, diluent or carrier. Pharmaceutical compositions can be prepared by methods well known in the art (e.g., Remington: The Science and Practice of Pharmacy, 22nd ed. (2012), A. Loyd et al., Pharmaceutical Press).

In some embodiments, the present disclosure provides antibody and antibody drug conjugates for use in therapy. In some embodiments, the present disclosure provides antibody and antibody drug conjugates for use in the treatment of a medical condition. In some embodiments, the medical condition is cancer, a cardiovascular disease, an autoimmune disease, or a neurodegenerative disease.

In further embodiments, the present disclosure provides the use of an antibody or antibody drug conjugate in the manufacture of a medicament for the treatment of cancer, a cardiovascular disease, an autoimmune disease, or a neurodegenerative disease.

In further embodiments, the present disclosure provides a method of administering a therapeutically effective amount of an antibody or antibody drug conjugate as disclosed herein, wherein the antibody or antibody drug conjugate is administered intravenously. In other embodiments, the present disclosure provides a method of administering a therapeutically effective amount of an antibody or antibody drug conjugate as disclosed herein, wherein the antibody or antibody drug conjugate is administered subcutaneously.

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

The term “antibody conjugate” or “conjugated antibody” or “antibody drug conjugate,” as used interchangeably herein, refers to a complex of an antibody or a fragment thereof, which fragment can comprise an antigen-binding fragment of the antibody or a constant domain fragment of the antibody thereof, and a non-antibody molecule. In one embodiment, the antibody and the non-antibody molecule are connected by a linker.

The term “non-antibody molecule,” as used herein, refers to a molecule that is not an immunoglobulin molecule. In one embodiment, the non-antibody molecule is a peptide comprising 2 or more amino acid residues. In a further embodiment, the non-antibody molecule is a small molecule drug. In a further embodiment, the non-antibody molecule is an oligonucleotide.

In a further embodiment, the oligonucleotide is an RNA oligonucleotide. In a further embodiment, the oligonucleotide is an RNAi oligonucleotide. In a further embodiment, the oligonucleotide is a double stranded oligonucleotide. In a further embodiment, the oligonucleotide is chemically modified.

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. The constant region refers to a region of an antibody, which comprises the Fc region and CH1 domain of the antibody heavy chain. Each light chain is comprised of a light chain variable region (VL) and a light chain constant region. The IgG isotype may be further divided into subclasses (e.g., IgG1, IgG2, IgG3, and IgG4). The numbering of the amino acid residues in the constant region is based on the EU index as in Kabat. Kabat et al, Sequences of Proteins of Immunological Interest, 5th edition, Bethesda, MD: U.S. Dept. of Health and Human Services, Public Health Service, National Institutes of Health (1991). The term EU index numbering or EU numbering is used interchangeably herein.

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). The North CDR definitions are used for the antibodies that specifically bind human IL-4Rα described herein.

Embodiments of the present disclosure also include antibody fragments or antigen binding fragments, such as Fab, Fab′, F(ab′)2, Fv fragments, scFv, scFab, disulfide-linked Fvs (sdFv), a Fd fragment, which may be for example, fused to an Fc region or an IgG heavy chain constant region.

The term “Fc region” as used herein, refers to a region of an antibody, which comprises the CH2 and CH3 domains of the antibody heavy chain. Optionally, the Fc region may include a portion of the hinge region or the entire hinge region of the antibody heavy chain. Biological activities such as effector function are attributable to the Fc region, which vary with the antibody isotype. Examples of antibody effector functions include, Fc receptor binding, antibody-dependent cell mediated cytotoxicity (ADCC), antibody-dependent cell mediated phagocytosis (ADCP), C1q binding, complement dependent cytotoxicity (CDC), phagocytosis, down regulation of cell surface receptors (e.g. B cell receptor); and B cell activation.

A “wild-type” antibody as referred to herein, is an antibody comprising the natural heavy chain constant region, including a natural Fc, and a natural light chain constant region of a human IgG1, IgG2, IgG3 or IgG4 antibody.

The terms “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

The terms “nucleic acid” or “polynucleotide” as used interchangeably herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

The term “subject” as used herein, refers to a mammal, including, but are not limited to, a human, chimpanzee, ape, monkey, cattle, horse, sheep, goat, swine, rabbit, dog, cat, rat, mouse, guinea pig, and the like. Preferably, the subject is a human.

The term “inhibits” as used herein, refers to for example, a reduction, lowering, slowing, decreasing, stopping, disrupting, abrogating, antagonizing, or blocking of a biological response or activity, but does not necessarily indicate a total elimination of a biological response.

The term “treatment” or “treating” as used herein, refers to all processes wherein there may be a slowing, controlling, delaying or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The term “about” as used herein, means within 5%.

As used herein, the term “a”, “an”, “the”, and similar terms used in the context of the present disclosure (especially in the context of the claims) are to be construed to cover both the singular and plural unless otherwise indicated herein or clearly contradicted by the context.

### BRIEF DESCRIPTION OF THE DRAWINGS

FIG. 1 show amino acid sequence alignment for human IgG1, IgG2, IgG3, and IgG4 heavy chain CH1-CH2-CH3 domains, highlighting the GNKRQ amino acid residues.

FIG. 2 compares the differential scanning calorimetry (DSC) thermogram of human IgG4P antibody variants with and without the KRQ amino acid substitution in the HC CH1 and CH2 domains.

FIG. 3 shows the KRQ amino acid substitutions in the heavy chain CH1 and CH2 domains of an IgG4P antibody do not impact C1q Elisa binding affinity relative to the IgG4P antibody lacking the KRQ amino acid substitutions.

FIG. 4 shows the KRQ amino acid substitutions in the heavy chain CH1 and CH2 domains of an IgG4P antibody do not impact ADCC activity relative to the IgG4P antibody lacking the KRQ amino acid substitutions

FIG. 5 shows the KRQ amino acid substitutions in the heavy chain CH1 and CH2 domains of an IgG4P antibody do not impact CDC activity relative to the IgG4P antibody lacking the KRQ amino acid substitutions.

FIGS. 6A-6B show the KRQ amino acid substitutions in the heavy chain CH1 and CH2 domains of an IgG4P antibody do not impact cell binding in B cells (6A) or monocytic cells (6B) relative to the IgG4P antibody lacking the KRQ amino acid substitutions.

### EXAMPLES

### Example 1: Antibody Generation and Engineering

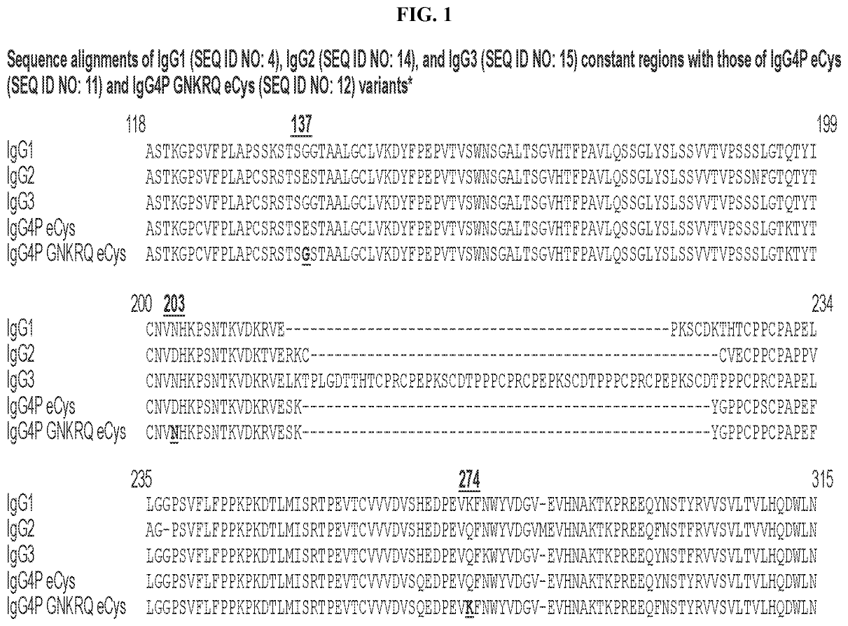

Antibody engineering, expression, and purification: Exemplified antibody Molecules 1, 2, 3, 4, 5, 6, and 7 were engineered in the heavy chain (HC) constant domains to improve viscosity through charge balancing to mitigate potential electrostatic interaction between the Fab and Fc. The HC CH1, CH2 and CH3 domains of a human IgG4 antibody when compared to the HC constant domains of a human IgG1, have lower isoelectric points (pI), due to an uneven charge distribution (Table 1). Five amino acid residues in the CH1, CH2, and CH3 domains of an IgG4 were identified as impacting the charge balance: 1) E137 (CH1 domain), 2) D203 (CH1 domain), 3) Q274 (CH2 domain), 4) Q355 (CH3 domain), and 5) E419 (CH3 domain). Alignment of the human IgG4 HC constant region with a human IgG1 HC constant region (FIG. 1) showed that the analogous position of these amino acid residues are different in the human IgG1 HC constant region, and were found to impact the overall pI of each domain. Alignment of the human IgG1, IgG2, IgG3, and IgG4 HC constant regions (FIG. 1) also showed that the analogous position of the GNKRQ amino acids were different at certain positions.

To match the pI for both the CH2 and CH3 domains of an IgG4 antibody to an IgG1 antibody and to minimize the potential introduction of an immunogenic peptide, the residues at the three of the five identified positions in the IgG4 Fc were converted to the corresponding residue found in an IgG1 wild-type Fc. The amino acid residue substitutions included: a positively charged lysine substituted for the neutrally charged glutamine at position 274 (Q274K), a positively charged arginine substituted for the neutrally charged glutamine at position 355 (Q355R) and a neutrally charged glutamine substituted for the negatively charged glutamic acid at position 419 (E419Q). The resulting IgG4 Fc was termed “KRQ”. To match the pI for the CH1 domain of the IgG4 to that of the IgG1, residues at positions 137 and 203 were converted to the corresponding residue found in an IgG1 wild-type CH1 domain. The amino acid substitutions included: a neutral charged glycine substituted for the negatively charged glutamic acid at position 137 (E137G) and a polar asparagine substituted for the negatively charged aspartic acid at positions 204 (D203N). The resulting IgG4 Fc having all 5 amino acid substitutions was termed “GNKRQ”.

Additionally, a double mutant comprising the Q355R and E419Q amino acid substitutions which only focuses on the CH3 domain termed “RQ”, and a single mutant comprising the Q274K amino acid substitution focused on the CH2 domain were also assessed.

To generate variants for Molecules 1 to 6, two version of the IgG4 Fc were utilized: 1) IgG4 Fc having a S228P amino acid residue substitution, which stabilizes the hinge and prevent arm exchange, was termed “IgG4P”, or 2) IgG4 Fc having the S228P amino acid residue substitution in combination with F234A and L235A amino acid substitutions, which are known to minimize effector function, termed “IgG4PAA”. The variable domains of each of Molecule 1 to 6 were cloned into the IgG4PAA Fc, the IgG4PAA KRQ Fc, the IgG4PAA GNKRQ Fc, and the wild type IgG1 Fc to generate the respective antibody variants for each of the 6 Molecules. For Molecule 7, variable domains were cloned into the IgG4P Fc, IgG4P KRQ Fc, and the IgG4PAA KRQ Fc. Molecule 7 variants also included engineered cysteines at positions 124 and 378 of the heavy chain (herein termed “eCys”) which were used as attachment points for small molecules in the generation of antibody drug conjugates (ADCs). For each of Molecules 1 to 7, a wild type CH1 domain of the human IgG4 and IgG1 was used respectively in the wild type and KRQ variants, while the light chain variable domains were cloned onto the human kappa constant region for all the molecules and the matching pairs were expressed together to generate the required intact antibodies for viscosity and biophysical assessment (see FIG. 1—IgG4P GNKRQ eCys).

Molecule 1 to 7 antibody variants were synthesized, expressed, and purified by well-known methods in the art, for example, an appropriate host cell, such as Chinese hamster ovarian cells (CHO), was either transiently or stably transfected with an expression system for secreting antibodies using a predetermined HC: LC vector ratio if two vectors were used, or a single vector system encoding both heavy chain and light chain. Clarified media, into which the antibody was secreted, was purified using commonly known techniques. HC constant domain and hinge sequences for the Molecule 1 to 7 antibody variants are shown in Table 2. Theoretical pIs (pI is the pH where the molecules net charge is neutral) for each heavy chain domain with and without the amino acid substitutions were calculated and compared using software analysis methods well known in the art (all calculated without a C-terminal lysine, which often undergoes post-translational clipping).

5 The results as demonstrated in Table 1 show that the theoretical pIs of the individual heavy chain CH1, CH2, and CH3 domains in the hIgG4 antibody variants increased to values similar to the pIs of the respective IgG1 domains, as a result of the amino acid substitution introduced into the IgG4 HC constant regions, when compared to the wild-type IgG4.

TABLE 1
Effect of constant domain amino acid substitutions on theoretical pI

| | | | | pI CH2, | pI CH1, |
|---|---|---|---|---|---|
| Isotype | pI CH1 | pI CH2 | pI CH3 | CH3 | CH2, CH3 |
| hIgG1 | 9.4 | 8.0 | 5.9 | 6.6 | 8.5 |
| hIgG4 | 8.8 | 7.0 | 5.2 | 5.8 | 6.7 |
| hIgG4 K | 8.8 | 8.0 | 5.2 | 6.0 | 7.2 |
| hIgG4 RQ | 8.8 | 7.0 | 5.9 | 6.3 | 7.7 |
| hIgG4 KRQ | 8.8 | 8.0 | 5.9 | 6.6 | 8.0 |
| hIgG4 GNKRQ | 9.3 | 8.0 | 5.9 | 6.6 | 8.4 |

TABLE 2
Antibody sequences of the HC constant region for Molecules 1 to 7

| | | Heavy chain |
|---|---|---|
| | | constant |
| | | region (CH1- |
| Antibody | | Hinge-CH2— |
| Molecule | | CH3 domains) |
| numbers | Antibody Isotype | sequence |
| Antibody Molecules 1-6 | hIgG1 | SEQ ID NO: 4 |
| Antibody Molecules 1-6 | hIgG4PAA | SEQ ID NO: 7 |
| Antibody Molecules 1-6 | hIgG4PAA KRQ | SEQ ID NO: 8 |
| Antibody Molecules 1-6 | hIgG4PAA GNKRQ | SEQ ID NO: 16 |
| Antibody Molecule 7 | hIgG4P KRQ eCys | SEQ ID NO: 9 |
| Antibody Molecule 7 | hIgG4P RQ eCys | SEQ ID NO: 10 |
| Antibody Molecule 7 | hIgG4P eCys | SEQ ID NO: 11 |
| Antibody Molecule 7 | hIgG4P GNKRQ eCys | SEQ ID NO: 12 |
| Antibody Molecule 7 | hIgG4P K eCys | SEQ ID NO: 13 |

### Example 2: Biophysical Properties of Exemplified Antibody Variants

Biophysical properties of the exemplified antibody variants for Molecules 1 to 7 were evaluated.

Intrinsic viscosity: Exemplified Molecules 1 to 6 antibody variants were concentrated to 130 mg/mL in 5 mM histidine, 280 mM mannitol, pH 6 buffer (H6M), and Molecule 7 variants were concentrated to 125 mg/mL. Protein concentrations were prepared and quantified neat using a variable-pathlength SoloVPE instrument (CTech). The intrinsic viscosity for each antibody was measured using VROC® initium (RheoSense) at 15° C. using the average of 9 replicate measurement. A minimum of 35 μL was used with 27 μL sample draw, which was injected onto a B05 chip with the system set to automatic testing mode, which adjusted the flow rate to achieve pressure readings of 50% of full scale.

The results as demonstrated in Table 3, show that the IgG4PAA KRQ antibody variants for Molecules 1, 3, 4, and 6 had a significant reduction in viscosity ranging from about 74% to about 30%, when compared to the respective IgG4PAA variant which lacked the KRQ amino acid substitutions. Furthermore, surprisingly, 5 of the 6 IgG4PAA KRQ antibody variants (for Molecules 2, 3, 4, 5, 6) demonstrated a reduction in viscosity when compared to the respective IgG1 variants. Particularly, the IgG4PAA KRQ variants for Molecules 2, 3, and 4 showed a reduction in viscosity ranging from about 82% to about 48% when compared to the respective IgG1 variants. Further improvement in viscosity for Molecules 1, 3, 4 and 6 was observed with the addition of the E137G and D204N mutations in the CH1 domain when compared to the respective IgG4PAA variant which lacked the KRQ amino acid substitutions. For Molecules 2 and 5 where the initial viscosities on the IgG4PAA were low prior to the substitutions, no benefit with the addition of the CH1 substitutions was observed when compared to the KRQ substitutions. However, surprisingly, 5 of the 6 IgG4PAA GNKRQ antibody variants (Molecules 1, 2, 3, 4, 6) demonstrated a reduction in viscosity when compared to the respective IgG1 variants. Particularly, the IgG4PAA KRQ variants for Molecules 3, 4 and 6 showed a reduction in viscosity ranging from about 83% to about 55% when compared to the respective IgG1 variants.

Additionally, the results as demonstrated in Table 4, show Molecule 7 IgG4P GNKRQ eCys (9.6 cP), IgG4P KRQ eCys (11.6 cP), IgG4P RQ eCys (14.9 cP), and IgG4P K eCys (21.0 cP) antibody variants had a reduction in viscosity from about 78% to about 51% respectively, thus showing an additive effect of the combination of the mutations, when compared to molecule 7 IgG4P eCys (43 cP) variant lacking any of the GNKRQ amino acid substitutions.

TABLE 3
Intrinsic viscosity of Molecules 1 to 6 antibody variants

| | Intrinsic Viscosity (cP) | | | |
|---|---|---|---|---|
| Molecule | IgG4PAA | IgG4PAA | | |
| # | GNKRQ | KRQ | IgG4PAA | IgG1 |
| 1 | 8.0 | 16.94 | 27.42 | 8.89 |
| 2 | 12.7 | 7.23 | 10.57 | 14.4 |
| 3 | 9.8 | 7.74 | 29.4 | 41.8 |
| 4 | 9.8 | 11.36 | 34.24 | 21.8 |
| 5 | 18.9 | 5.42 | 5.94 | 6.94 |
| 6 | 14 | 76.88 | 167.52 | 83.76 |

TABLE 4
Intrinsic viscosity of Molecule 7 IgG4P antibody variants

| | Intrinsic Viscosity (cP) | | | | |
|---|---|---|---|---|---|
| | IgG4P | IgG4P | IgG4P | | |
| | GNKRQ | KRQ | RQ | IgG4P | IgG4P |
| | eCys | eCys | eCys | K eCys | eCys |
| Molecule 7 | 9.6 | 11.6 | 14.9 | 21.0 | 43 |

cP = Centipoise

Intrinsic viscosity of Molecule 7 antibody variants in an antibody drug conjugate format: The intrinsic viscosity of Molecule 7 antibody variants IgG4P KRQ eCys, IgG4P GNKRQ and IgG4P eCys were tested in an antibody drug conjugate format. Briefly, engineered cysteines at positions 124 and 378 in the heavy chain of Molecule 7 IgG4P eCys, IgG4P KRQ eCys and IgG4P GNKRQ antibody variants were used to generate antibody drug conjugates using a lipophilic small molecule with a short linker. The conjugation was done through a short reduction in the presence of the reducing agent dithiothreitol, followed by a desalting step to remove the reducing agent and a short oxidation step in the present of dehydroascorbic acid. The reduction step and subsequent desalting step removed any cysteinylation on the engineered cysteines at positions 124 and 378 of the heavy chain, which occurs during cell culture expression. The oxidation step reformed the native disulfide bonds which includes two disulfide pairs in the hinge and 1 disulfide pair between the heavy and light chain. The small molecule was then conjugated to the antibody using maleimide based chemistry which reacts with the free thiols of the engineered cysteine residues. Viscosity was measured, essentially as described above.

The results as demonstrated in Table 5, show that Molecule 7 IgG4P KRQ eCys ADC (12.2 cP) and Molecule 7 IgG4P GNKRQ eCys ADC (10.2 cP) had a 48-fold and a 58-fold reduction in viscosity respectively when compared to Molecule 7 IgG4P eCys ADC (592 cP), which lacks the GNKRQ amino acid residue substitutions, thus showing an additive effect of the combination of the mutations.

TABLE 5
Intrinsic viscosity of Molecule 7
IgG4P KRQ antibody drug conjugate

| | Intrinsic Viscosity (cP) |
|---|---|
| Molecule 7-IgG4P eCys-ADC | 592 |
| Molecule 7-IgG4P KRQ | 12.2 |
| eCys-ADC | |
| Molecule 7-IgG4P GNKRQ | 10.2 |
| eCys-ADC | |

cP = Centipose

Thermal Stability: Differential Scanning calorimetry (DSC) was used to evaluate the stability of exemplified Molecules 1 to 7 variants against thermal denaturation. DSC was run using a Malvern MircoCal VP-DSC instrument. Samples were heated from 20° C. to 110° C. at a constant rate of 60° C./hour. Analysis methods were performed using the MicroCal VP-Capillary DSC Automated Analysis program. Baseline corrections were performed, and T onset and TM1 were determined. Although the transition temperatures for unfolding of the 3 domains, including the CH2, CH3 and Fab were not well resolved for either the IgG4P eCys or the IgG4P KRQ eCys the thermal melting temperatures of Molecule 7 IgG4P eCys and IgG4P KRQ eCys antibody variants were measured in PBS, pH 7.2 buffer.

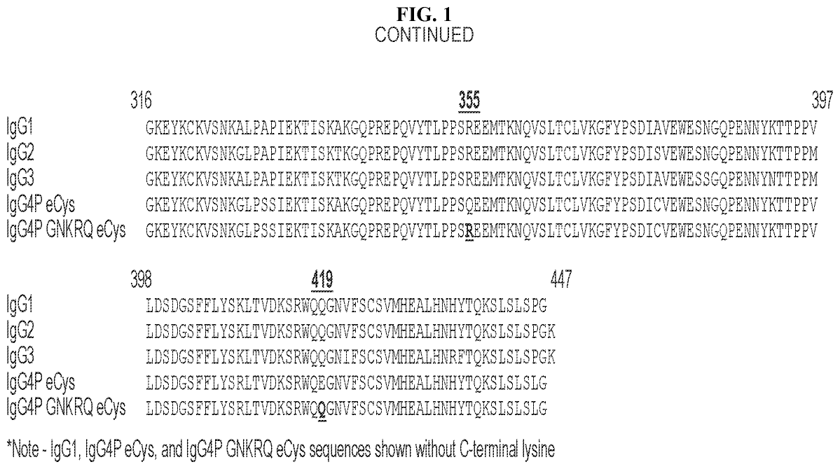

The results as demonstrated in Table 6 and FIG. 2, show that introducing amino acid residues KRQ into the CH2 and CH3 of the IgG4 constant domain of the antibody did not negatively impact the thermal stability of the resulting antibody. Onset of thermal aggregation (Tagg): Intrinsic fluorescence (IF) and light scattering measurements were performed using the Prometheus Panta (Nano Temper Technologies) equipped with high sensitivity capillaries. Samples were prepared at 0.5 mg/mL in PBS, pH 7.2 buffer and the onset of aggregation (Tagg) was determined using a thermal ramp from 20-95° C. at a constant rate of 1° C./min. Using PR Panta Analysis (X64) software (V1.0.2), the onset of aggregation was determined using a 2-state fit based on the backreflection signal at 385 nm with a threshold of 0.5% increase from baseline.

The results as demonstrated in Table 6, show that the onset of aggregation was comparable between Molecule 7 IgG4P KRQ eCys and IgG4P eCys antibody variants, and thus showing that introducing amino acid residues KRQ into the CH2 and CH3 of the IgG4 constant domain of the antibody did not negatively impact the thermal stability of the resulting antibody.

TABLE 6
Thermal aggregation for Molecule
7 IgG4P KRQ eCys antibody variant

| | Tonset | TM1 | Tagg |
|---|---|---|---|
| Molecule 7-IgG4P eCys | 59.0 | 69.8 | 67.2 |
| Molecule 7-IgG4P KRQ eCys | 59.1 | 69.9 | 67 |

All values in degrees celsius (° C.)

Aggregation upon temperature stress: The solution stability of the exemplified Molecule variants over time was assessed at approximately 100 mg/mL in a common 5 mM histidine pH 6.0 buffer with excipients. Concentrated samples were incubated for a period of 4 weeks at 5° C. and 35° C., respectively. Following incubation, samples were analyzed for the percentage of high molecular weight (% HMW) species with size exclusion chromatography (SEC).

The results as demonstrated in Table 7, show that the KRQ amino acid residue substitutions did not significantly impact aggregation over a 4-week time period at either 5° C. or 35° C.; specifically, the results showed that the high concentration solution stability of the IgG4P KRQ eCys antibody was comparable to the IgG4P eCys antibody variant.

Freeze/thaw stability was assessed using a 3 repeated slow, controlled temperature cycle which mimics the freeze/thaw conditions of large volumes of bulk drug substance placed at −70° C. Samples were assessed in both 5 mM histidine, 280 mM mannitol, 0.05% PS80, pH 6 (H6MT) and 5 mM histidine, 280 mM sucrose, 0.05% PS80, pH 6 (H6ST). Following incubation, samples were analyzed for the change in percentage of high molecular weight (4% HMW) species with analytical size exclusion chromatography (aSEC). Samples were also visually inspected for phase separation or precipitate formation.

The results as demonstrated in Table 7, show that introducing amino acid residues KRQ into the CH2 and CH3 of the IgG4 constant domain of the antibody did not impact the freeze/thaw stability of the resulting antibodies. Specifically, Molecule 7 IgG4P KRQ eCys antibody variant exhibited Δ3.4% HMW in H6MT and Δ0.5% HMW in H6ST which is comparable to the IgG4P eCys antibody which exhibited Δ2.5% HMW in H6MT and Δ0.3% HMW in H6ST as measured by aSEC after the 3 freeze-thaw cycles. Visual observation did not show any phase separation or precipitation for either the IgG4P KRQ eCys or the IgG4P eCys antibody variants.

Solubility: Solubility was assessed by concentrating 100 mg of the antibody variants with a 30 kDa molecular weight cut-off centrifugal filter (for example, Amicon U.C. filters, Millipore, catalog #UFC903024) to a volume of approximately 0.5 mL. The final concentration of the sample was measured by UV absorbance at 280 nm using a Solo VPE spectrophotometer (C Technologies, Inc). Following incubation, samples were analyzed for the percentage of high molecular weight (% HMW) species with analytical size exclusion chromatography (aSEC). Samples were also visually inspected for phase separation or precipitate formation.

The results as demonstrated in Table 7, show that the Molecule 7 IgG4P KRQ eCys antibody variant exhibit Δ0.6% HMW is comparable to the Molecule 7 IgG4P eCys antibody which exhibited Δ0.4% HMW as measured by aSEC. Visual observation did not show any phase separation or precipitation for either the IgG4P KRQ eCys or the IgG4P eCys.

TABLE 7
Molecular parameters for Molecule 7
IgG4P and IgG1 antibody variants

| | Molecule 7 | Molecule 7 |
|---|---|---|
| | IgG4P | IgG4P KRQ |
| | eCys | eCys |
| tCHO titer (g/L) | 1.1 | 0.9 |
| % HMW post-protein A | 1.2 | 1 |
| chromatography | | |
| % HMW after 4-week | 0.1 | 0.2 |
| incubation at 5° C. | | |
| % HMW after 4-week | 2.4 | 2.2 |
| incubation at 35° C. | | |
| Freeze/Thaw H6MT | 2.4 | 3.4 |
| (Δ % HMW) | | |
| Freeze/Thaw H6ST | 0.3 | 0.5 |
| (Δ % HMW) | | |
| Solubility (Δ % HMW) | 0.4 | 0.6 |

The biophysical analysis demonstrated that introducing amino acid residues KRQ into the CH2 and CH3 domains of an IgG4 significantly reduced viscosity without negatively impacting aggregation, solubility, or thermal stability of the antibody when compared to the IgG4P antibody lacking the KRQ amino acid substitutions.

### Example 3. Effector Function Activity

In vitro Fcγ receptor binding, ADCC, and CDC assays were conducted to evaluate whether the amino acid substitutions in the Fc regions of the antibodies altered Fc function.

Human Fcγ Receptor Binding: The binding affinity of Molecule 7 IgG4 antibody variants to human Fcγ receptors was evaluated by surface plasmon resonance (SPR) analysis. Biacore T100 (Cytiva), Biacore reagents, and Scrubber2 Biacore Evaluation Software (Biologics 2008) were used for the SPR analysis A series S CM5 chip (Cytiva P/N BR100530) was prepared using the manufacturer's EDC/NHS amine coupling method (Cytiva P/N BR100050). Briefly, the surfaces of all 4 flow cells (FC) were activated by injecting a 1:1 mixture of EDC/NHS for 7 minutes at 10 μL/minute. Protein A (Calbiochem P/N 539202) was diluted to 100 μg/mL in 10 mM acetate, pH 4.5 buffer, and immobilized for approximately 4000 RU onto all 4 FCs by 7 minute injection at a flow rate of 10 μL/minute. Unreacted sites were blocked with a 7 minute injection of ethanolamine at 10 μL/minute. Injections of 2× 10 μL of glycine, pH 1.5, was used to remove any noncovalently associated protein. Running buffer was 1×HBS-EP+ (TEKNOVA, P/N H8022). The FcγR extracellular domains (ECDs)-FcγRI (CD64), FcγRIIA_131R, and FcγRIIA_131H (CD32a), FcγRIIIA_158V, FcγRIIIA_158F (CD16a), and FcγRIIb (CD32b) were produced from stable CHO cell expression, and purified using IgG Sepharose and size exclusion chromatography. For FcγRI binding, antibodies were diluted to 2.5 μg/mL in running buffer, and approximately 150 RU of each antibody was captured in FCs 2 through 4 (RU captured). FC1 was the reference FC, therefore no antibody was captured in FC1. FcγRI ECD was diluted to 200 nM in running buffer and then two-fold serially diluted in running buffer to 0.78 nM. Duplicate injections of each concentration were injected over all FCs at 40 L/minute for 120 seconds followed by a 1200 second dissociation phase. Regeneration was performed by injecting 15 μL of 10 mM glycine, pH 1.5, at 30 μL/minute over all FCs. Reference-subtracted data was collected as FC2 FC1, FC3-FC1, and FC4-FC1 and the measurements were obtained at 25° C. The affinity (KD) was calculated using either steady state equilibrium analysis with the Scrubber 2 Biacore Evaluation Software or a “1:1 (Langmuir) binding” model in BIA Evaluation. For FcγRIIa, FcγRIIb, and FcγRIIIa binding, antibodies were diluted to 5 μg/mL in running buffer, and approximately 500 RU of each antibody was captured in FCs 2 through 4 (RUcaptured). FC1 was again the reference FC. Fcγ receptor ECDs were diluted to 10 UM in running buffer and then serially diluted 2-fold in running buffer to 39 nM. Duplicate injections of each concentration were injected over all FCs at 40 μL/minute for 60 seconds followed by a 120 second dissociation phase. Regeneration was performed by injecting 15 μL of 10 mM glycine, pH 1.5, at 30 μL/minutes over all FCs. Reference-subtracted data was collected as FC2-FC1, FC3-FC1, and FC4-FC1, and the measurements were obtained at 25° C. The affinity (KD) was calculated using the steady state equilibrium analysis with the Scrubber 2 Biacore Evaluation Software. Each receptor was assayed at least two times.

The results as demonstrated in Table 8, show that the Molecule 7 IgG4P KRQ eCys and IgG4P eCys antibody variants had comparable binding affinity to each of the Fcγ receptors, thus showing that introducing amino acid residues KRQ into the CH2 and CH3 of the IgG4 constant domain of the antibody, did not impact the Fc binding activity of the resulting antibody.

TABLE 8
Binding affinities of Molecule 7 IgG4P antibody
variants to human Fcγ receptors

| | Molecule 7- | | Molecule 7- | |
|---|---|---|---|---|
| | IgG4P KRQ eCys | | IgG4P eCys | |
| | Average KD | Std Dev | Average KD | Std Dev |
| Fcγ RI | 442.3 pM | 21.4 | 306.0 pM | 18.4 |
| Fcγ RIIA_131H | 3.75 μM | 0.11 | 3.73 μM | 0.04 |
| Fcγ RIIA_131R | 1.67 μM | 0.06 | 1.51 μM | 0.01 |
| Fcγ RIIb | 2.05 μM | 0.2 | 1.69 μM | 0.04 |
| Fcγ RIIIA_158V | 6.22 μM | 0.8 | 3.93 μM | 0.28 |
| Fcγ RIIIA_158F | >10 μM | — | >10 μM | — |

C1q Binding: Binding of Molecule 7 IgG4 antibody variants to human C1q was evaluated by Elisa. A 96-well microplate was coated with 100 μL/well of Molecule 7 IgG4 KRQ eCys antibody variant diluted in DPBS (Dulbecco's HyClone) from 10 μg/mL to 0.19 μg/mL, and incubated overnight at 4° C. The coating reagent was removed, plate was blocked with 200 μL/well casein blocking buffer (Thermo) and incubated for 2 hours at room temperature (RT). The plate was washed 3 times with wash buffer (1×TBE with 0.05% Tween 20), and 10 μg/mL Human C1q (MS Biomedical) diluted in casein blocking reagent is added at 100 L/well and incubated for 3 hours at RT. Humanized IgG1 and humanized IgG4P isotype control antibodies were used as positive and negative controls respectively. The plate was then washed three times with wash buffer and 100 μL/well of a 1:800 times dilution of sheep anti-human C1q-HRP (Abcam #ab46191) in casein blocker was added and incubated for 1 hour at RT. The plate was then washed 6 times with wash buffer, and 100 μL/well of TMB Substrate (Pierce) was added to each well and incubated for 7 minutes. 100 μL/well of 1 N HCl was added to stop the reaction. Optical density was immediately measured at 450 nm on a colorimetric microplate reader. Data was analyzed using SoftMax Pro 7.1 Data Acquisition and Analysis Software.

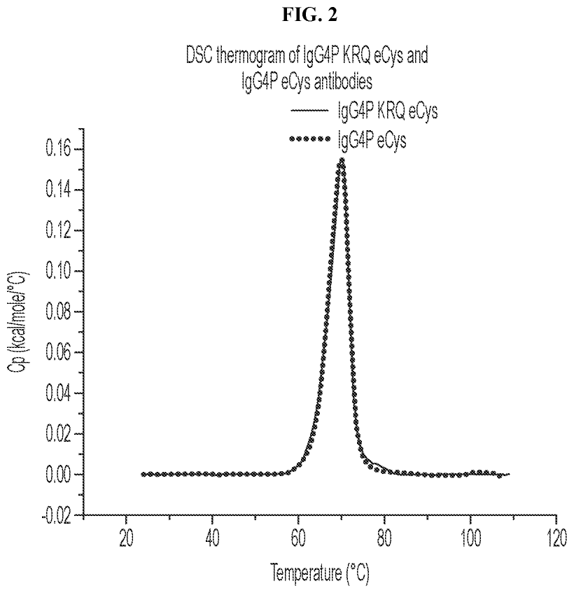

The results as demonstrated in FIG. 3, shows that the Molecule 7 IgG4P KRQ eCys antibody variant had comparable C1q binding to the IgG4P control, thus showing that introducing amino acid residues KRQ into the CH2 and CH3 of the IgG4 constant domain of the antibody, did not impact complement binding of the resulting antibody. Antibody dependent cellular cytotoxicity (ADCC): In vitro ADCC assays of the Molecule 7 antibody variants was evaluated with a reporter gene based ADCC assay.

Briefly, Daudi cells (ATCC, #CCL-213) and human CD20 target cell lines and Jurkat cells expressing functional FcγRIIIa (V158)-NFAT-Luc (Eli Lilly and Company) as the effector cell line were used. All test antibody variants and cells were diluted in assay medium containing RPMI-1640 (no phenol red) with 0.1 mM non-essential amino acids (NEAA), 1 mM sodium pyruvate, 2 mM L-glutamine, 500 U/mL of penicillin-streptomycin, and 0.1% w/v BSA. Test antibodies were first diluted to a 3× concentration of 3.3 μg/mL and then serially diluted 7 times in a 1:4 ratio. 50 μL/well of each antibody was aliquoted in duplicate in white opaque bottom 96-well plate (Costar, #3917). CD20 antibody was used as a positive control. Daudi target cells were then added to the plates at 5×10⁴ cells/well in 50 μL aliquots, and incubated for 1 hour at 37° C. Next, Jurkat V158 cells were added to the wells at 150,000 cells/well in 50 μL aliquots and incubated for 4 hours at 37° C., followed by addition of 100 μL/well of One-Glo Luciferase substrate (Promega, #E8130). The contents of the plates were mixed using a plate shaker at low speed, incubated at room temperature for 5 minutes, and the luminescence signal was read on a BioTek microplate reader (BioTek Instruments) using 0.2 cps integration. Data was analyzed using GraphPad Prism 9 and the relative luminescence units (RLU) for each antibody concentration were plotted in a scatter format of antibody concentration versus RLU. Results were representative of two independent experiments.

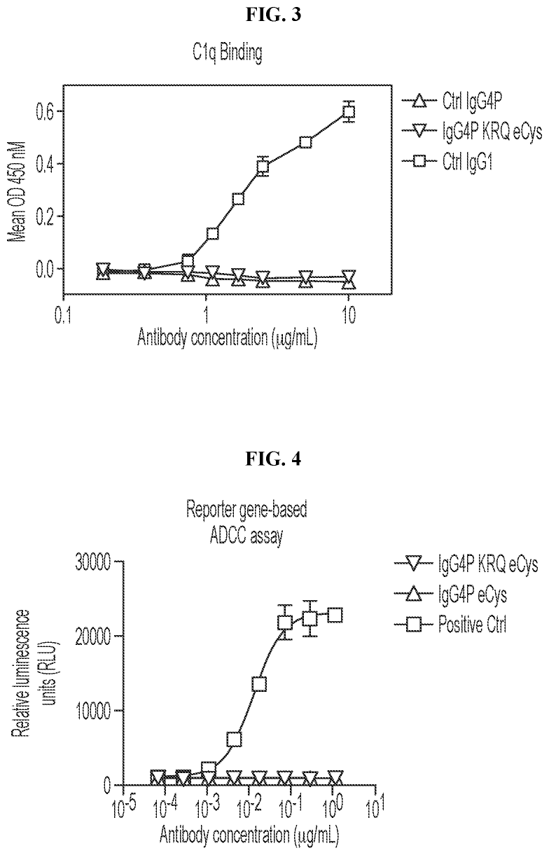

The results as demonstrated in FIG. 4, show that the Molecule 7 antibody variants IgG4P eCys and IgG4P KRQ eCys had comparable ADCC activity (i.e., did not induce ADCC activity) in the reporter gene based ADCC assay, thus showing that introducing amino acid residues KRQ into the CH2 and CH3 of the IgG4 constant domain of the antibody did not impact effector function activity of the resulting antibody. The positive control CD20 antibody showed potent ADCC activity.

Complement dependent cellular cytotoxicity (CDC): In vitro CDC assays of the Molecule 7 IgG4 antibody variants were conducted using Daudi cells (ATCC, #CCL-213). All test antibodies, complement, and cells were diluted in assay medium consisting of RPMI-1640 (no phenol red) with 0.1 mM non-essential amino acids (NEAA), 1 mM sodium pyruvate, 2 mM L-glutamine, 500 U/mL of penicillin-streptomycin, and 0.1% w/v BSA. Test antibodies were first diluted to a 3× concentration of 100 μg/mL and then serially diluted 7 times in a 1:4 ratio. 50 μL/well of each antibody were aliquoted in duplicate in white opaque bottom 96-well plate (Costar, #3917). Daudi target cells were then added at 50,000 cells/well in 50 μL aliquots, with the exemplified antibodies, and CD20 positive control antibody were incubated for 1 hour at 37° C. Next, human serum complement (Quidel, #A113) quickly thawed in a 37° C. water bath was diluted 1:6 in assay medium and added at 50 μL/well to the assay plate. The plate was incubated for 2 hours at 37° C., followed by the addition of 100 μL/well CellTiter Glo substrate (Promega, #G7571) to each well. The contents of the plates were mixed using a plate shaker at low speed, incubated at room temperature for 5 minutes, and the luminescence signal was read on a BioTek microplate reader (BioTek Instruments) using 0.2 cps integration. Data was analyzed using GraphPad Prism v9 and the relative luminescence units (RLU) for each antibody concentration were plotted in a scatter format of antibody concentration versus RLU.

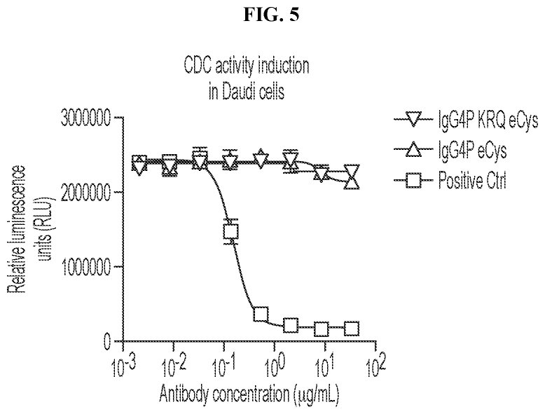

The results as demonstrated in FIG. 5, show Molecule 7 IgG4P eCys and IgG4P KRQ eCys antibody variants had comparable CDC activity in Daudi cells, thus showing that introducing the amino acid residues KRQ into the CH2 and CH3 of the IgG4 constant domain of the antibody did not impact CDC activity of the of the resulting antibody. The CDC assay positive control CD20 antibody showed potent CDC activity.

The results of the Fcγ, C1q binding assays, and the cell-based effector function ADCC and CDC assays showed that introduction of the KRQ amino acid residues into the constant domains of the antibody variants did not impact the Fc function of the antibodies.

### Example 4. Cell Binding of Variants

Binding to B Cells and Monocytic Cells: Binding of the exemplified Molecule 7 IgG4P antibody variants to B cells and monocytic cells was tested in a Fluorescence Activated Cell Sorting (FACS) assay. Human PBMCs were isolated from human blood samples by standard Ficoll-Paque™ plus (GE HEALTHCARE) density gradient centrifugation methods. Freshly isolated cells PBMCs were resuspended at 2 million cells/mL and allowed to rest for 15 minutes at room temperature, then plated at 100 μL/well into a round bottom 96-well plate (COSTAR®) and were washed with FACS buffer (PBS containing 2% fetal bovine serum from Corning®). Exemplified IgG4P antibody variants conjugated to Alexa Fluor® 647 according to manufacturer's protocol (Thermo Fisher Scientific) were added to the wells at 66.67 nM and diluted 4-fold in duplicate. Equivalent volume of 2× antibody cocktail containing: Human TruStain FcX™, FITC anti-human CD3 Antibody, Alexa Fluor® 700 anti-human CD4 Antibody (all from Biolegend®), CD20 Monoclonal Antibody (2H7), PerCP-Cyanine5.5 (Thermo Fisher Scientific) and CD14 PE-Cy™7 Mouse Anti-Human CD14 (BD Biosciences) was then added to the wells. Cells were incubated at 4° C. for 30 minutes, then washed twice with FACS buffer and resuspended in a final volume of 100 μL FACS buffer. Viability dye, Sytox™ blue (Thermo Fisher Scientific), was added and the samples were analyzed via a flow cytometer (LSRFortessa™ X-20; BD BIOSCIENCES). Data analysis was performed using FlowJo software and statistical analysis is performed using GraphPad Prism 9. Data represented the mean±SEM of the percentage of positive cells from the CD20 B cell and CD3/CD20 negative, CD14 positive monocytic cell populations from two donors. Curves were generated by fitting a sigmoidal curve of the log (Ab concentration) vs. the percent of Ab positive expressing cells from the individual cell populations.

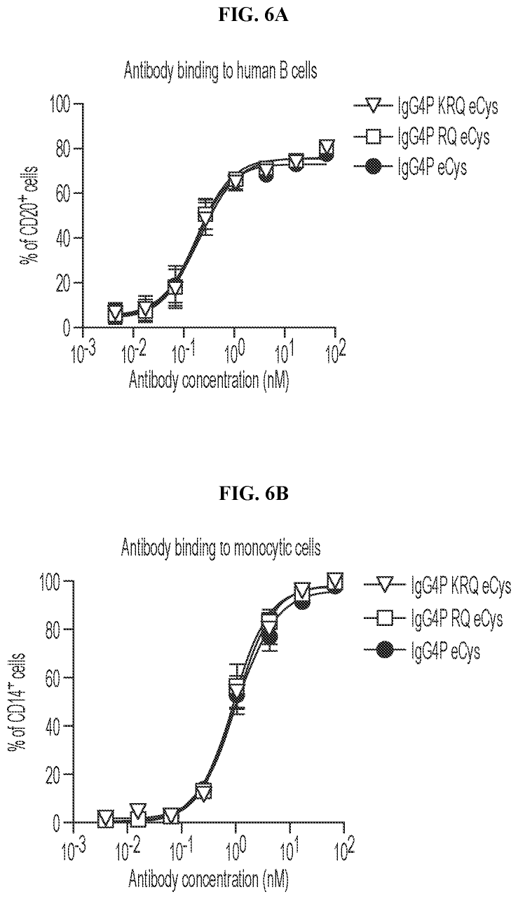

The results as demonstrated in Table 9 and FIGS. 6A and 6B, show that the exemplified Molecule 7 antibody variants IgG4P KRQ eCys, IgG4P RQ eCys, and IgG4P eCys bound with comparable affinity to the PBMC isolated B cells (EC₅₀ of 0.20 nM, 0.18 nM and 0.17 nM, respectively) and monocytic cells (EC₅₀ of 1.04 nM, 0.90 nM, and 1.00 nM, respectively), thus showing that introducing the amino acid residues KRQ or RQ into the CH2 and CH3 of the IgG4 constant domain of the antibody did not impact binding of the variable regions of the resulting antibodies.

TABLE 9
Binding of Molecule 7 IgG4P variant
antibodies to B cells and monocytic cells

| | B Cells | Monocytic Cells |
|---|---|---|
| Antibody | EC₅₀ (nM) | EC₅₀ (nM) |
| Molecule 7-IgG4P KRQ eCys | 0.20 | 1.04 |
| Molecule 7-IgG4P RQ eCys | 0.18 | 0.90 |
| Molecule 7-IgG4P eCys | 0.17 | 1.00 |

SEQUENCE LISTING
Human Kappa Constant (for Molecules
1, 2, 3, 4, 5, 6, 7)
SEQ ID NO: 1
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC
Human IgG1 CH1 (for Molecules 1 2,
3, 4, 5, 6)
SEQ ID NO: 2
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV
Human IgG1 Hinge (for Molecules 1,
2, 3, 4, 5, 6)
SEQ ID NO: 3
EPKSCDKTHTCPPCP
Human IgG1 CH1-Hinge-CH2-CH3
(for Molecules 1, 2, 3, 4, 5, 6)
SEQ ID NO: 4
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
Human IgG4 CH1 (for Molecules 1,
2, 3, 4, 5, 6)
SEQ ID NO: 5
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRV
Human IgG4 (S228P) Hinge (for
Molecules 1, 2, 3, 4, 5, 6, 7)
SEQ ID NO: 6
ESKYGPPCPPCP
Human IgG4PAA (S228P, F234A, L235A)
CH1-Hinge-CH2-CH3 (for Molecules 1,
2, 3, 4, 5, 6)
SEQ ID NO: 7
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEAAGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLG
Human IgG4PAA KRQ (S228P, Q274K,
F234A, L235A, Q355R, E419Q) CH1-
Hinge-CH2-CH3 (for Molecules 1,
2, 3, 4, 5, 6)
SEQ ID NO: 8
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEAAGGPSVFLFPPKPKDTLMISRTPEVTCVVVDVSQED
PEVKFNWYVDGVEVHNAKTKPREEQFNSTYRVVSVLTVLHQDWLNGKEYK
CKVSNKGLPSSIEKTISKAKGQPREPQVYTLPPSREEMTKNQVSLTCLVK
GFYPSDIAVEWESNGQPENNYKTTPPVLDSDGSFFLYSRLTVDKSRWQQG
NVFSCSVMHEALHNHYTQKSLSLSLG
Human IgG4P KRQ eCys (S124C, S228P,
Q274K, Q355R, A378C, E419Q) CH1-
Hinge-CH2-CH3 (for Molecule 7)
SEQ ID NO: 9
ASTKGPCVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVKFNWYVDGVEVHNAKTKPREEQFNSTYRVVSVLTVLHQDWLNGKEYK
CKVSNKGLPSSIEKTISKAKGQPREPQVYTLPPSREEMTKNQVSLTCLVK
GFYPSDICVEWESNGQPENNYKTTPPVLDSDGSFFLYSRLTVDKSRWQQG
NVFSCSVMHEALHNHYTQKSLSLSLG
Human IgG4P RQ eCys (S124C, S228P,
Q355R, A378C, E419Q) CH1-Hinge-CH2-
CH3 (for Molecule 7)
SEQ ID NO: 10
ASTKGPCVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSREEMTKNQVSLTCLVK
GFYPSDICVEWESNGQPENNYKTTPPVLDSDGSFFLYSRLTVDKSRWQQG
NVFSCSVMHEALHNHYTQKSLSLSLG
Human IgG4P eCys (S124C, S228P, A378C)
CH1-Hinge-CH2-CH3 (for Molecule 7)
SEQ ID NO: 11
ASTKGPCVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDICVEWESNGQPENNYKTTPPVLDSDGSFFLYSRLTVDKSRWQEG
NVFSCSVMHEALHNHYTQKSLSLSLG
Human IgG4P GNKRQ eCys (S124C, E137G,
D203N, S228P, Q274K, Q355R, A378C, E419Q)
CH1-Hinge-CH2-CH3 (for Molecule 7)
SEQ ID NO: 12
ASTKGPCVFPLAPCSRSTSGSTAALGCLVKDYFPEPVTVSWNSGALTSGV
HTFPAVLQSSGLYSLSSVVTVPSSSLGTKTYTCNVNHKPSNTKVDKRVES
KYGPPCPPCPAPEFLGGPSVFLFPPKPKDTLMISRTPEVTCVVVDVSQED
PEVKFNWYVDGVEVHNAKTKPREEQFNSTYRVVSVLTVLHQDWLNGKEYK
CKVSNKGLPSSIEKTISKAKGQPREPQVYTLPPSREEMTKNQVSLTCLVK
GFYPSDICVEWESNGQPENNYKTTPPVLDSDGSFFLYSRLTVDKSRWQQG
NVFSCSVMHEALHNHYTQKSLSLSLG
Human IgG4P K eCys (S124C, S228P,
Q274K, A378C) CH1-Hinge-CH2-CH3
(for Molecule 7)
SEQ ID NO: 13
ASTKGPCVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVKFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDICVEWESNGQPENNYKTTPPVLDSDGSFFLYSRLTVDKSRWQEG
NVFSCSVMHEALHNHYTQKSLSLSLG
Human IgG2 CH1-Hinge-CH2-CH3
SEQ ID NO: 14
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVMEVHNAKTKPREEQFNSTFRVVSVLTVVHQDWLNGKEYK
CKVSNKGLPAPIEKTISKTKGQPREPQVYTLPPSREEMTKNQVSLTCLVK
GFYPSDISVEWESNGQPENNYKTTPPMLDSDGSFFLYSKLTVDKSRWQQG
NVFSCSVMHEALHNHYTQKSLSLSPGK
Human IgG3 CH1-Hinge-CH2-CH3
SEQ ID NO: 15
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgG4PAA GNKRQ (S228P, E137G,
D203N, Q274K, F234A, L235A, Q355R,
E419Q) CH1-Hinge-CH2-CH3 (for
Molecules 1, 2, 3, 4, 5, 6)
SEQ ID NO: 16
ASTKGPSVFPLAPCSRSTSGSTAALGCLVKDYFPEPVTVSWNSGALTSGV
HTFPAVLQSSGLYSLSSVVTVPSSSLGTKTYTCNVNHKPSNTKVDKRVES
KYGPPCPPCPAPEAAGGPSVFLFPPKPKDTLMISRTPEVTCVVVDVSQED
PEVKFNWYVDGVEVHNAKTKPREEQFNSTYRVVSVLTVLHQDWLNGKEYK
CKVSNKGLPSSIEKTISKAKGQPREPQVYTLPPSREEMTKNQVSLTCLVK
GFYPSDIAVEWESNGQPENNYKTTPPVLDSDGSFFLYSRLTVDKSRWQQG
NVFSCSVMHEALHNHYTQKSLSLSLG

1. An antibody comprising a human IgG heavy chain constant region comprising a CH1, a CH2, and a CH3 domain, and a human IgG light chain constant region, wherein the human IgG heavy chain constant region comprises:
a lysine at amino acid residue 274 (EU numbering) of the CH2 domain;
an arginine at amino acid residue 355 (EU numbering) of the CH3 domain;
a glutamine at amino acid residue 419 (EU numbering) of the CH3 domain;
a glycine at amino acid residue 137 (EU numbering) of the CH1 domain;
an asparagine at amino acid residue 203 (EU numbering) of the CH1 domain;
a lysine at amino acid residue 274 (EU numbering) of the CH2 domain and an arginine at amino acid residue 355 (EU numbering) of the CH3 domain;
a lysine at amino acid residue 274 (EU numbering) of the CH2 domain and a glutamine at amino acid residue 419 (EU numbering) of the CH3 domain;
an arginine at amino acid residue 355 (EU numbering) of the CH3 domain and a glutamine at amino acid residue 419 (EU numbering) of the CH3 domain;
a lysine at amino acid residue 274 (EU numbering) of the CH2 domain and a glycine at amino acid residue 137 (EU numbering) of the CH1 domain;
a lysine at amino acid residue 274 (EU numbering) of the CH2 domain and an asparagine at amino acid residue 203 (EU numbering) of the CH1 domain;
an arginine at amino acid residue 355 (EU numbering) of the CH3 domain and a glycine at amino acid residue 137 (EU numbering) of the CH1 domain;
an arginine at amino acid residue 355 (EU numbering) of the CH3 domain and an asparagine at amino acid residue 203 (EU numbering) of the CH1 domain;
a glutamine at amino acid residue 419 (EU numbering) of the CH3 domain and a glycine at amino acid residue 137 (EU numbering) of the CH1 domain;
a glutamine at amino acid residue 419 (EU numbering) of the CH3 domain and an asparagine at amino acid residue 203 (EU numbering) of the CH1 domain;
a glycine at amino acid residue 137 (EU numbering) of the CH1 domain and an asparagine at amino acid residue 203 (EU numbering) of the CH1 domain;
a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain;
a lysine at amino acid residue 274 (EU numbering) of the CH2 domain, an arginine at amino acid residue 355 (EU numbering) of the CH3 domain, and a glycine at amino acid residue 137 (EU numbering) of the CH1 domain;
a lysine at amino acid residue 274 (EU numbering) of the CH2 domain, an arginine at amino acid residue 355 (EU numbering) of the CH3 domain, and an asparagine at amino acid residue 203 (EU numbering) of the CH1 domain;
a lysine at amino acid residue 274 (EU numbering) of the CH2 domain, a glycine at amino acid residue 137 (EU numbering) of the CH1 domain, and an asparagine at amino acid residue 203 (EU numbering) of the CH1 domain;
a lysine at amino acid residue 274 (EU numbering) of the CH2 domain, a glutamine at amino acid residue 419 (EU numbering) of the CH3 domain, and a glycine at amino acid residue 137 (EU numbering) of the CH1 domain;
a lysine at amino acid residue 274 (EU numbering) of the CH2 domain, a glutamine at amino acid residue 419 (EU numbering) of the CH3 domain, and an asparagine at amino acid residue 203 (EU numbering) of the CH1 domain;
an arginine at amino acid residue 355 (EU numbering) of the CH3 domain, a glutamine at amino acid residue 419 (EU numbering) of the CH3 domain, and a glycine at amino acid residue 137 (EU numbering) of the CH1 domain;
an arginine at amino acid residue 355 (EU numbering) of the CH3 domain, a glutamine at amino acid residue 419 (EU numbering) of the CH3 domain, and an asparagine at amino acid residue 203 (EU numbering) of the CH1 domain;
an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain;
a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain;
a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and a glycine at amino acid residue 137 (EU numbering) of the CH1 domain;
a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and an asparagine at amino acid residue 203 (EU numbering) of the CH1 domain;
a lysine at amino acid residue 274 (EU numbering) of the CH2 domain, an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain;
a lysine at amino acid residue 274 (EU numbering) of the CH2 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain;
an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain; or
a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain.

2. The antibody of claim 1, comprising a human IgG heavy chain constant region comprising a CH1, a CH2, and a CH3 domain, and a human IgG light chain constant region, wherein the human IgG heavy chain constant region comprises:
a lysine at amino acid residue 274 (EU numbering) of the CH2 domain;
an arginine at amino acid residue 355 (EU numbering) of the CH3 domain;
a glutamine at amino acid residue 419 (EU numbering) of the CH3 domain;
a lysine at amino acid residue 274 (EU numbering) of the CH2 domain and an arginine at amino acid residue 355 (EU numbering) of the CH3 domain;
a lysine at amino acid residue 274 (EU numbering) of the CH2 domain and a glutamine at amino acid residue 419 (EU numbering) of the CH3 domain;
an arginine at amino acid residue 355 (EU numbering) of the CH3 domain and a glutamine at amino acid residue 419 (EU numbering) of the CH3 domain; or
a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

3. The antibody of claim 2, wherein the human IgG heavy chain constant region comprises:
a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

4. The antibody of any one of claim 1, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3.

5. The antibody of any one of claim 1, wherein the antibody is a human IgG2, human IgG3, or human IgG4 antibody.

6. The antibody of claim 5, wherein the antibody is a human IgG4 antibody.

7. An antibody comprising a human IgG heavy chain constant region comprising any one of SEQ ID NOs: 8, 9, 10, 12, 13, or 16.

8. An antibody drug conjugate comprising the antibody of claim 1.

9. The antibody of claim 1, wherein the antibody has reduced viscosity as compared to a wild-type antibody comprising a wild-type human IgG heavy chain constant region.

10. The antibody of claim 9, wherein the viscosity is reduced by about 25 percent to about 80 percent when compared to the wild-type antibody.

11. A method of reducing viscosity of a human IgG4 antibody comprising, generating a variant of the human IgG4 antibody comprising a modified human IgG4 heavy chain constant region comprising a CH1, a CH2, and a CH3 domain, and a human IgG light chain constant region, wherein the modified human IgG4 heavy chain constant region comprises the following amino acid residues:
a lysine at amino acid residue 274 (EU numbering) of the CH2 domain;
an arginine at amino acid residue 355 (EU numbering) of the CH3 domain;
a glutamine at amino acid residue 419 (EU numbering) of the CH3 domain;
a glycine at amino acid residue 137 (EU numbering) of the CH1 domain;
an asparagine at amino acid residue 203 (EU numbering) of the CH1 domain;
a lysine at amino acid residue 274 (EU numbering) of the CH2 domain and an arginine at amino acid residue 355 (EU numbering) of the CH3 domain;
a lysine at amino acid residue 274 (EU numbering) of the CH2 domain and a glutamine at amino acid residue 419 (EU numbering) of the CH3 domain;
an arginine at amino acid residue 355 (EU numbering) of the CH3 domain and a glutamine at amino acid residue 419 (EU numbering) of the CH3 domain;
a lysine at amino acid residue 274 (EU numbering) of the CH2 domain and a glycine at amino acid residue 137 (EU numbering) of the CH1 domain;
a lysine at amino acid residue 274 (EU numbering) of the CH2 domain and an asparagine at amino acid residue 203 (EU numbering) of the CH1 domain;
an arginine at amino acid residue 355 (EU numbering) of the CH3 domain and a glycine at amino acid residue 137 (EU numbering) of the CH1 domain;
an arginine at amino acid residue 355 (EU numbering) of the CH3 domain and an asparagine at amino acid residue 203 (EU numbering) of the CH1 domain;
a glutamine at amino acid residue 419 (EU numbering) of the CH3 domain and a glycine at amino acid residue 137 (EU numbering) of the CH1 domain;
a glutamine at amino acid residue 419 (EU numbering) of the CH3 domain and an asparagine at amino acid residue 203 (EU numbering) of the CH1 domain;
a glycine at amino acid residue 137 (EU numbering) of the CH1 domain and an asparagine at amino acid residue 203 (EU numbering) of the CH1 domain;
a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain;
a lysine at amino acid residue 274 (EU numbering) of the CH2 domain, an arginine at amino acid residue 355 (EU numbering) of the CH3 domain, and a glycine at amino acid residue 137 (EU numbering) of the CH1 domain;
a lysine at amino acid residue 274 (EU numbering) of the CH2 domain, an arginine at amino acid residue 355 (EU numbering) of the CH3 domain, and an asparagine at amino acid residue 203 (EU numbering) of the CH1 domain;
a lysine at amino acid residue 274 (EU numbering) of the CH2 domain, a glycine at amino acid residue 137 (EU numbering) of the CH1 domain, and an asparagine at amino acid residue 203 (EU numbering) of the CH1 domain;
a lysine at amino acid residue 274 (EU numbering) of the CH2 domain, a glutamine at amino acid residue 419 (EU numbering) of the CH3 domain, and a glycine at amino acid residue 137 (EU numbering) of the CH1 domain;
a lysine at amino acid residue 274 (EU numbering) of the CH2 domain, a glutamine at amino acid residue 419 (EU numbering) of the CH3 domain, and an asparagine at amino acid residue 203 (EU numbering) of the CH1 domain;
an arginine at amino acid residue 355 (EU numbering) of the CH3 domain, a glutamine at amino acid residue 419 (EU numbering) of the CH3 domain, and a glycine at amino acid residue 137 (EU numbering) of the CH1 domain;
an arginine at amino acid residue 355 (EU numbering) of the CH3 domain, a glutamine at amino acid residue 419 (EU numbering) of the CH3 domain, and an asparagine at amino acid residue 203 (EU numbering) of the CH1 domain;
an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain;
a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain;
a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and a glycine at amino acid residue 137 (EU numbering) of the CH1 domain;
a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, and an asparagine at amino acid residue 203 (EU numbering) of the CH1 domain;
a lysine at amino acid residue 274 (EU numbering) of the CH2 domain, an arginine at amino acid residue 355 (EU numbering) of the CH3 domain, a glycine at amino acid residue 137 (EU numbering) of the CH1 domain, and an asparagine at amino acid residue 203 (EU numbering) of the CH1 domain;
a lysine at amino acid residue 274 (EU numbering) of the CH2 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain;
an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain; or
a lysine at amino acid residue 274 (EU numbering) of the CH2 domain, an arginine at amino acid residue 355 (EU numbering) of the CH3 domain, a glutamine at amino acid residue 419 (EU numbering) of the CH3 domain, a glycine at amino acid residue 137 (EU numbering) of the CH1 domain, and an asparagine at amino acid residue 203 (EU numbering) of the CH1 domain.

12. The method of claim 11, wherein the modified human IgG4 heavy chain constant region comprises the following amino acid residues:
a lysine at amino acid residue 274 (EU numbering) of the CH2 domain;
an arginine at amino acid residue 355 (EU numbering) of the CH3 domain;
a glutamine at amino acid residue 419 (EU numbering) of the CH3 domain;
a lysine at amino acid residue 274 (EU numbering) of the CH2 domain and an arginine at amino acid residue 355 (EU numbering) of the CH3 domain;
a lysine at amino acid residue 274 (EU numbering) of the CH2 domain and a glutamine at amino acid residue 419 (EU numbering) of the CH3 domain;
an arginine at amino acid residue 355 (EU numbering) of the CH3 domain and a glutamine at amino acid residue 419 (EU numbering) of the CH3 domain; or
a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

13. The method of claim 11, wherein the modified human IgG4 heavy chain constant region comprises the following amino acid residues:
a lysine at amino acid residue 274 (EU numbering) of the CH2 domain, an arginine at amino acid residue 355 (EU numbering) of the CH3 domain, and a glutamine at amino acid residue 419 (EU numbering) of the CH3 domain.

14. The method of claim 11, wherein the antibody comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3.

15. The method of claim 11, further comprising generating an antibody drug conjugate comprising the variant human IgG4 antibody.

16. The method of claim 11, wherein the antibody has reduced viscosity as compared to the wild-type human IgG4 antibody.

17. The method of claim 16, wherein the viscosity is reduced by about 25 percent to about 80 percent when compared to the wild-type human IgG4 antibody.

18. A nucleic acid comprising a sequence encoding the heavy chain or light chain of the antibody of claim 1.

19. A vector comprising the nucleic acid of claim 18.

20. A cell comprising the vector of claim 19.

21. The cell of claim 20, wherein the cell is a mammalian cell.

22. A process of producing an antibody comprising culturing the cell of claim 21, under conditions such that the antibody is expressed and recovering the expressed antibody from the culture medium.

23. An antibody produced by the process of claim 22.

24. A pharmaceutical composition comprising the antibody of claim 1, and a pharmaceutically acceptable excipient, diluent or carrier.

25. A method of administering the antibody of claim 1 to a subject in need thereof, wherein the antibody is administered subcutaneously to the subject.


SEQUENCE LISTING (16 sequences)

SEQ ID NO: 1 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 15 aa, Homo sapiens
EPKSCDKTHTCPPCP

SEQ ID NO: 4 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 6 - protein, 12 aa, synthetic construct
ESKYGPPCPPCP

SEQ ID NO: 7 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 9 - protein, 326 aa, synthetic construct
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDICVEWESNGQPENNYKTTPPVLDSDGSFFLYSRLTVDKSRWQQGNVFSCSVMHEALHNHYTQKSLSLSLG

SEQ ID NO: 10 - protein, 326 aa, synthetic construct
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDICVEWESNGQPENNYKTTPPVLDSDGSFFLYSRLTVDKSRWQQGNVFSCSVMHEALHNHYTQKSLSLSLG

SEQ ID NO: 11 - protein, 326 aa, synthetic construct
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 12 - protein, 326 aa, synthetic construct
ASTKGPCVFPLAPCSRSTSGSTAALGCLVKDYFPEPVTVSWNSGALTSGVHTFPAVLQSSGLYSLSSVVTVPSSSLGTKTYTCNVNHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDICVEWESNGQPENNYKTTPPVLDSDGSFFLYSRLTVDKSRWQQGNVFSCSVMHEALHNHYTQKSLSLSLG

SEQ ID NO: 13 - protein, 326 aa, synthetic construct
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 16 - protein, 326 aa, synthetic construct
ASTKGPSVFPLAPCSRSTSGSTAALGCLVKDYFPEPVTVSWNSGALTSGVHTFPAVLQSSGLYSLSSVVTVPSSSLGTKTYTCNVNHKPSNTKVDKRVESKYGPPCPPCPAPEAAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG